(19) United States
(12) Patent Application Publication
(10) Pub. No.: US 2023/0017411 A1
(43) Pub. Date: Jan. 19, 2023

(54) ENDOSCOPE WITH SOURCE AND PIXEL LEVEL IMAGE MODULATION FOR MULTISPECTRAL IMAGING

(71) Applicant: Cilag GmbH International, Zug (CH)

(72) Inventors: Tarik Yardibi, Wayland, MA (US); Emir Osmanagic, Norwell, MA (US); Patrick J. Treado, Pittsburgh, PA (US); Shawna K. Tazik, Kansas City, MO (US); Matthew P. Nelson, Harrison City, PA (US); Jeffrey Beckstead, Valencia, PA (US)

(21) Appl. No.: 17/375,615

(22) Filed: Jul. 14, 2021

(51) Int. Cl. A61B 1/06; A61B 1/00; A61B 1/05; A61B 5/00

(52) U.S. Cl. CPC A61B 1/0638; A61B 1/0005; A61B 1/00186; A61B 1/05; A61B 5/489; A61B 5/4893

(57) ABSTRACT

A system may be provided which comprises an illumination source adapted to simultaneously illuminate a surgical site with spectral light comprising a first wavelength of light and a second wavelength of light, a first set of sensors comprising sensors adapted to detect visible light, and a second set of sensors comprising sensors adapted to detect the first wavelength of light; and sensors adapted to detect the second wavelength of light. In such a case, the system may also comprise a display coupled to a processor configured to display an enhanced image of the surgical site comprising a visible light image from data detected by the first set of sensors and an overlay identifying a target structure based on light detected by the second set of sensors while the surgical site is illuminated by spectral light comprising the first and second wavelengths of light.

### BACKGROUND

Surgical systems may incorporate an imaging system, which may allow the clinician(s) to view the surgical site and/or one or more portions thereof on one or more displays such as a monitor. The display(s) may be local and/or remote to a surgical theater. An imaging system may include a scope with a camera that views the surgical site and transmits the view to a display that is viewable by the clinician. Scopes include, but are not limited to, laparoscopes, robotic laparoscopes, arthroscopes, angioscopes, bronchoscopes, choledochoscopes, colonoscopes, cytoscopes, duodenoscopes, enteroscopes, esophagogastro-duodenoscopes (gastroscopes), endoscopes, laryngoscopes, nasopharyngo-neproscopes, sigmoidoscopes, thoracoscopes, ureteroscopes, and exoscopes. Imaging systems may be limited by the information that they are able to recognize and/or convey to the clinician(s). For example, certain concealed structures, physical contours, and/or dimensions within a three-dimensional space may be unrecognizable intraoperatively by certain imaging systems. Additionally, certain imaging systems may be incapable of communicating and/or conveying certain information to the clinician(s) intraoperatively.

Examples of surgical imaging systems are disclosed in U.S. Pat. Pub. No. 2020/0015925, entitled “Combination Emitter and Camera Assembly,” published Jan. 16, 2020; U.S. Pat. Pub. No. 2020/0015899, entitled “Surgical Visualization with Proximity Tracking Features,” published Jan. 16, 2020; U.S. Pat. Pub. No. 2020/0015924, entitled “Robotic Light Projection Tools,” published Jan. 16, 2020; and U.S. Pat. Pub. No. 2020/0015898, entitled “Surgical Visualization Feedback System,” published Jan. 16, 2020. The disclosure of each of the above-cited U.S. patents and patent applications is incorporated by reference herein.

While various kinds of surgical instruments and systems have been made and used, it is believed that no one prior to the inventor(s) has made or used the invention described in the appended claims.

### BRIEF DESCRIPTION OF THE DRAWINGS

The accompanying drawings, which are incorporated in and constitute a part of this specification, illustrate embodiments of the invention, and, together with the general description of the invention given above, and the detailed description of the embodiments given below, serve to explain the principles of the present invention.

FIG. 1 depicts a schematic view of an exemplary surgical visualization system including an imaging device and a surgical device;

FIG. 2 depicts a schematic diagram of an exemplary control system that may be used with the surgical visualization system of FIG. 1;

FIG. 3 depicts a schematic diagram of another exemplary control system that may be used with the surgical visualization system of FIG. 1;

FIG. 4 depicts exemplary hyperspectral identifying signatures to differentiate anatomy from obscurants, and more particularly depicts a graphical representation of a ureter signature versus obscurants;

FIG. 5 depicts exemplary hyperspectral identifying signatures to differentiate anatomy from obscurants, and more particularly depicts a graphical representation of an artery signature versus obscurants;

FIG. 6 depicts exemplary hyperspectral identifying signatures to differentiate anatomy from obscurants, and more particularly depicts a graphical representation of a nerve signature versus obscurants;

FIG. 7A depicts a schematic view of an exemplary emitter assembly that may be incorporated into the surgical visualization system of FIG. 1, the emitter assembly including a single electromagnetic radiation (EMR) source, showing the emitter assembly in a first state;

FIG. 7B depicts a schematic view of the emitter assembly of FIG. 7A, showing the emitter assembly in a second state;

7C Depicts a Schematic View of the Emitter Assembly of FIG. 7A, Showing the Emitter Assembly in a Third State;

FIG. 8 depicts a potential configuration for an imaging device;

FIG. 9 depicts an exemplary hyperspectral identifying signatures for distinguishing a target structure from background tissue;

FIG. 10 depicts an exemplary pixel configuration;

FIG. 11 depicts an exemplary configuration in which light sensors are disposed on separate optical paths; and

FIG. 12 depicts an exemplary pixel configuration.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

For clarity of disclosure, the terms “proximal” and “distal” are defined herein relative to a surgeon, or other operator, grasping a surgical device. The term “proximal” refers to the position of an element arranged closer to the surgeon, and the term “distal” refers to the position of an element arranged further away from the surgeon. Moreover, to the extent that spatial terms such as “top,” “bottom,” “upper,” “lower,” “vertical,” “horizontal,” or the like are used herein with reference to the drawings, it will be appreciated that such terms are used for exemplary description purposes only and are not intended to be limiting or absolute. In that regard, it will be understood that surgical instruments such as those disclosed herein may be used in a variety of orientations and positions not limited to those shown and described herein.

Furthermore, the terms “about,” “approximately,” and the like as used herein in connection with any numerical values or ranges of values are intended to encompass the exact value(s) referenced as well as a suitable tolerance that enables the referenced feature or combination of features to function for the intended purpose(s) described herein.

Similarly, the phrase “based on” should be understood as referring to a relationship in which one thing is determined at least in part by what it is specified as being “based on.” This includes, but is not limited to, relationships where one thing is exclusively determined by another, which relationships may be referred to using the phrase “exclusively based on.”

### I. Exemplary Surgical Visualization System

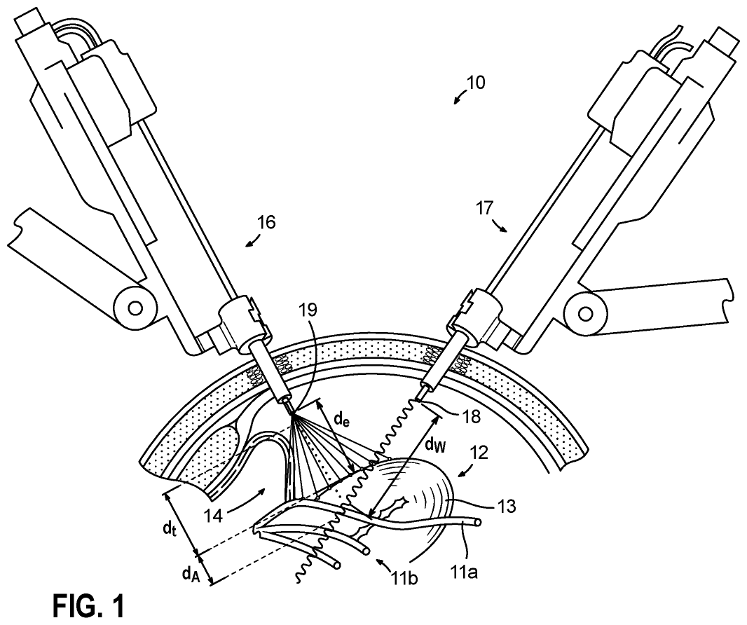

FIG. 1 depicts a schematic view of a surgical visualization system (10) according to at least one aspect of the present disclosure. The surgical visualization system (10) may create a visual representation of a critical structure (11a, 11b) within an anatomical field. The surgical visualization system (10) may be used for clinical analysis and/or medical intervention, for example. In certain instances, the surgical visualization system (10) may be used intraoperatively to provide real-time, or near real-time, information to the clinician regarding proximity data, dimensions, and/or distances during a surgical procedure. The surgical visualization system (10) is configured for intraoperative identification of critical structure(s) and/or to facilitate the avoidance of critical structure(s) (11a, 11b) by a surgical device. For example, by identifying critical structures (11a, 11b), a clinician may avoid maneuvering a surgical device into a critical structure (11a, 11b) and/or a region in a predefined proximity of a critical structure (11a, 11b) during a surgical procedure. The clinician may avoid dissection of and/or near a vein, artery, nerve, and/or vessel, for example, identified as a critical structure (11a, 11b), for example. In various instances, critical structure(s) (11a, 11b) may be determined on a patient-by-patient and/or a procedure-by-procedure basis.

Critical structures (11a, 11b) may be any anatomical structures of interest. For example, a critical structure (11a, 11b) may be a ureter, an artery such as a superior mesenteric artery, a vein such as a portal vein, a nerve such as a phrenic nerve, a common bile duct, a perfusion, and/or a cancerous or non-cancerous tumor, among other anatomical structures. In other instances, a critical structure (11a, 11b) may be any foreign structure in the anatomical field, such as a surgical device, surgical fastener, clip, tack, bougie, band, and/or plate, for example. In one aspect, a critical structure (11a, 11b) may be embedded in tissue. Stated differently, a critical structure (11a, 11b) may be positioned below a surface of the tissue. In such instances, the tissue conceals the critical structure (11a, 11b) from the clinician's view. A critical structure (11a, 11b) may also be obscured from the view of an imaging device by the tissue. The tissue may be fat, connective tissue, adhesions, and/or organs, for example. In other instances, a critical structure (11a, 11b) may be partially obscured from view. A surgical visualization system (10) is shown being utilized intraoperatively to identify and facilitate avoidance of certain critical structures, such as a ureter (11a) and vessels (11b) in an organ (12) (the uterus in this example), that are not visible on a surface (13) of the organ (12).

### A. Overview of Exemplary Surgical Visualization System

With continuing reference to FIG. 1, the surgical visualization system (10) incorporates tissue identification and geometric surface mapping in combination with a distance sensor system (14). In combination, these features of the surgical visualization system (10) may determine a position of a critical structure (11a, 11b) within the anatomical field and/or the proximity of a surgical device (16) to the surface (13) of the visible tissue and/or to a critical structure (11a, 11b). The surgical device (16) may include an end effector having opposing jaws (not shown) and/or other structures extending from the distal end of the shaft of the surgical device (16). The surgical device (16) may be any suitable surgical device such as, for example, a dissector, a stapler, a grasper, a clip applier, a monopolar RF electrosurgical instrument, a bipolar RF electrosurgical instrument, and/or an ultrasonic instrument. As described herein, a surgical visualization system (10) may be configured to achieve identification of one or more critical structures (11a, 11b) and/or the proximity of a surgical device (16) to critical structure(s) (11a, 11b).

The depicted surgical visualization system (10) includes an imaging system that includes an imaging device (17), such as a camera of a scope, for example, that is configured to provide real-time views of the surgical site. In various instances, an imaging device (17) includes a spectral camera (e.g., a hyperspectral camera, multispectral camera, a fluorescence detecting camera, or selective spectral camera), which is configured to detect reflected or emitted spectral waveforms and generate a spectral cube of images based on the molecular response to the different wavelengths. Views from the imaging device (17) may be provided to a clinician; and, in various aspects of the present disclosure, may be augmented with additional information based on the tissue identification, landscape mapping, and input from a distance sensor system (14). In such instances, a surgical visualization system (10) includes a plurality of subsystems—an imaging subsystem, a surface mapping subsystem, a tissue identification subsystem, and/or a distance determining subsystem. These subsystems may cooperate to intraoperatively provide advanced data synthesis and integrated information to the clinician(s).

The imaging device (17) of the present example includes an emitter (18), which is configured to emit spectral light in a plurality of wavelengths to obtain a spectral image of hidden structures, for example. The imaging device (17) may also include a three-dimensional camera and associated electronic processing circuits in various instances. In one aspect, the emitter (18) is an optical waveform emitter that is configured to emit electromagnetic radiation (e.g., near-infrared radiation (NIR) photons) that may penetrate the surface (13) of a tissue (12) and reach critical structure(s) (11a, 11b). The imaging device (17) and optical waveform emitter (18) thereon may be positionable by a robotic arm or a surgeon manually operating the imaging device. A corresponding waveform sensor (e.g., an image sensor, spectrometer, or vibrational sensor, etc.) on the imaging device (17) may be configured to detect the effect of the electromagnetic radiation received by the waveform sensor.

The wavelengths of the electromagnetic radiation emitted by the optical waveform emitter (18) may be configured to enable the identification of the type of anatomical and/or physical structure, such as critical structure(s) (11a, 11b). The identification of critical structure(s) (11a, 11b) may be accomplished through spectral analysis, photo-acoustics, fluorescence detection, and/or ultrasound, for example. In one aspect, the wavelengths of the electromagnetic radiation may be variable. The waveform sensor and optical waveform emitter (18) may be inclusive of a multispectral imaging system and/or a selective spectral imaging system, for example. In other instances, the waveform sensor and optical waveform emitter (18) may be inclusive of a photoacoustic imaging system, for example. In other instances, an optical waveform emitter (18) may be positioned on a separate surgical device from the imaging device (17). By way of example only, the imaging device (17) may provide hyperspectral imaging in accordance with at least some of the teachings of U.S. Pat. No. 9,274,047, entitled “System and Method for Gross Anatomic Pathology Using Hyperspectral Imaging,” issued Mar. 1, 2016, the disclosure of which is incorporated by reference herein in its entirety.

The depicted surgical visualization system (10) also includes an emitter (19), which is configured to emit a pattern of light, such as stripes, grid lines, and/or dots, to enable the determination of the topography or landscape of a surface (13). For example, projected light arrays may be used for three-dimensional scanning and registration on a surface (13). The projected light arrays may be emitted from an emitter (19) located on a surgical device (16) and/or an imaging device (17), for example. In one aspect, the projected light array is employed to determine the shape defined by the surface (13) of the tissue (12) and/or the motion of the surface (13) intraoperatively. An imaging device (17) is configured to detect the projected light arrays reflected from the surface (13) to determine the topography of the surface (13) and various distances with respect to the surface (13). By way of further example only, a visualization system (10) may utilize patterned light in accordance with at least some of the teachings of U.S. Pat. Pub. No. 2017/0055819, entitled “Set Comprising a Surgical Instrument,” published Mar. 2, 2017, the disclosure of which is incorporated by reference herein in its entirety; and/or U.S. Pat. Pub. No. 2017/0251900, entitled “Depiction System,” published Sep. 7, 2017, the disclosure of which is incorporated by reference herein in its entirety.

The depicted surgical visualization system (10) also includes a distance sensor system (14) configured to determine one or more distances at the surgical site. In one aspect, the distance sensor system (14) may include a time-of-flight distance sensor system that includes an emitter, such as the structured light emitter (19); and a receiver (not shown), which may be positioned on the surgical device (16). In other instances, the time-of-flight emitter may be separate from the structured light emitter (19). In one general aspect, the emitter portion of the time-of-flight distance sensor system (14) may include a laser source and the receiver portion of the time-of-flight distance sensor system (14) may include a matching sensor. A time-of-flight distance sensor system (14) may detect the “time of flight,” or how long the laser light emitted by the structured light emitter (19) has taken to bounce back to the sensor portion of the receiver. Use of a very narrow light source in a structured light emitter (19) may enable a distance sensor system (14) to determine the distance to the surface (13) of the tissue (12) directly in front of the distance sensor system (14).

Referring still to FIG. 1, a distance sensor system (14) may be employed to determine an emitter-to-tissue distance (dₑ) from a structured light emitter (19) to the surface (13) of the tissue (12). A device-to-tissue distance (dₜ) from the distal end of the surgical device (16) to the surface (13) of the tissue (12) may be obtainable from the known position of the emitter (19) on the shaft of the surgical device (16) relative to the distal end of the surgical device (16). In other words, when the distance between the emitter (19) and the distal end of the surgical device (16) is known, the device-to-tissue distance (dₜ) may be determined from the emitter-to-tissue distance (dₑ). In certain instances, the shaft of a surgical device (16) may include one or more articulation joints; and may be articulatable with respect to the emitter (19) and the jaws. The articulation configuration may include a multi-joint vertebrae-like structure, for example. In certain instances, a three-dimensional camera may be utilized to triangulate one or more distances to the surface (13).

As described above, a surgical visualization system (10) may be configured to determine the emitter-to-tissue distance (dₑ) from an emitter (19) on a surgical device (16) to the surface (13) of a uterus (12) via structured light. The surgical visualization system (10) is configured to extrapolate a device-to-tissue distance (dₜ) from the surgical device (16) to the surface (13) of the uterus (12) based on emitter-to-tissue distance (dₑ). The surgical visualization system (10) is also configured to determine a tissue-to-ureter distance (dA) from a ureter (11a) to the surface (13) and a camera-to-ureter distance (d_{w}), from the imaging device (17) to the ureter (11a). Surgical visualization system (10) may determine the camera-to-ureter distance (d_{w}), with spectral imaging and time-of-flight sensors, for example. In various instances, a surgical visualization system (10) may determine (e.g., triangulate) a tissue-to-ureter distance (dA) (or depth) based on other distances and/or the surface mapping logic described herein.

### B. First Exemplary Control System

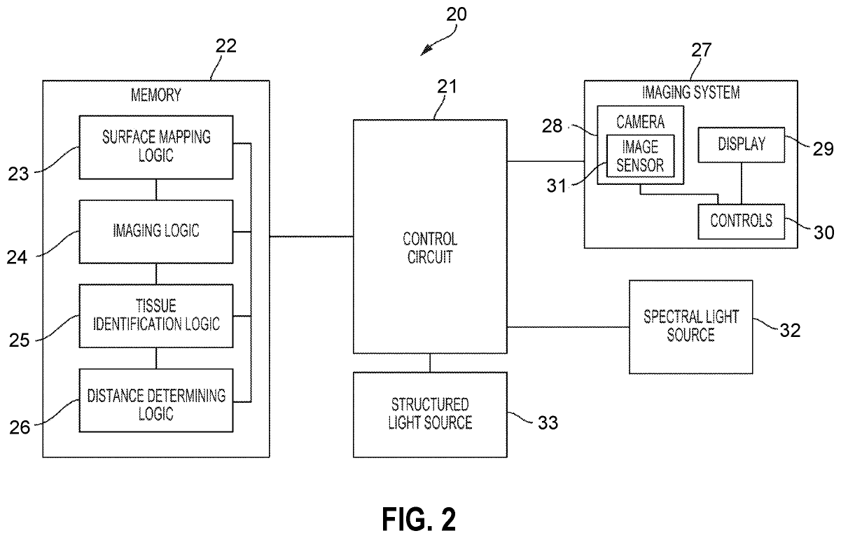

FIG. 2 is a schematic diagram of a control system (20), which may be utilized with a surgical visualization system (10). The depicted control system (20) includes a control circuit (21) in signal communication with a memory (22). The memory (22) stores instructions executable by the control circuit (21) to determine and/or recognize critical structures (e.g., critical structures (11a, 11b) depicted in FIG. 1), determine and/or compute one or more distances and/or three-dimensional digital representations, and to communicate certain information to one or more clinicians. For example, a memory (22) stores surface mapping logic (23), imaging logic (24), tissue identification logic (25), or distance determining logic (26) or any combinations of logic (23, 24, 25, 26). The control system (20) also includes an imaging system (27) having one or more cameras (28) (like the imaging device (17) depicted in FIG. 1), one or more displays (29), one or more controls (30) or any combinations of these elements. The one or more cameras (28) may include one or more image sensors (31) to receive signals from various light sources emitting light at various visible and invisible spectra (e.g., visible light, spectral imagers, three-dimensional lens, among others). The display (29) may include one or more screens or monitors for depicting real, virtual, and/or virtually-augmented images and/or information to one or more clinicians.

In various aspects, a main component of a camera (28) includes an image sensor (31). An image sensor (31) may include a Charge-Coupled Device (CCD) sensor, a Complementary Metal Oxide Semiconductor (CMOS) sensor, a short-wave infrared (SWIR) sensor, a hybrid CCD/CMOS architecture (sCMOS) sensor, and/or any other suitable kind(s) of technology. An image sensor (31) may also include any suitable number of chips.

The depicted control system (20) also includes a spectral light source (32) and a structured light source (33). In certain instances, a single source may be pulsed to emit wavelengths of light in the spectral light source (32) range and wavelengths of light in the structured light source (33) range. Alternatively, a single light source may be pulsed to provide light in the invisible spectrum (e.g., infrared spectral light) and wavelengths of light on the visible spectrum. A spectral light source (32) may include a hyperspectral light source, a multispectral light source, a fluorescence excitation light source, and/or a selective spectral light source, for example. In various instances, tissue identification logic (25) may identify critical structure(s) via data from a spectral light source (32) received by the image sensor (31) portion of a camera (28). Surface mapping logic (23) may determine the surface contours of the visible tissue based on reflected structured light. With time-of-flight measurements, distance determining logic (26) may determine one or more distance(s) to the visible tissue and/or critical structure(s) (11a, 11b). One or more outputs from surface mapping logic (23), tissue identification logic (25), and distance determining logic (26), may be provided to imaging logic (24), and combined, blended, and/or overlaid to be conveyed to a clinician via the display (29) of the imaging system (27).

### C. Second Exemplary Control System

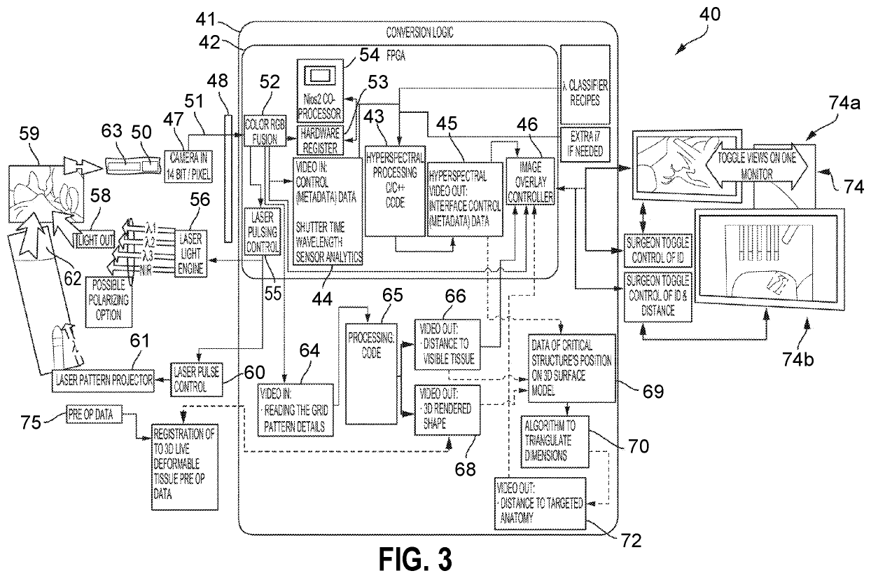

FIG. 3 depicts a schematic of another control system (40) for a surgical visualization system, such as the surgical visualization system (10) depicted in FIG. 1, for example. This control system (40) is a conversion system that integrates spectral signature tissue identification and structured light tissue positioning to identify critical structures, especially when those structures are obscured by other tissue, such as fat, connective tissue, blood, and/or other organs, for example. Such technology could also be useful for detecting tissue variability, such as differentiating tumors and/or non-healthy tissue from healthy tissue within an organ.

The control system (40) depicted in FIG. 3 is configured for implementing a hyperspectral or fluorescence imaging and visualization system in which a molecular response is utilized to detect and identify anatomy in a surgical field of view. This control system (40) includes a conversion logic circuit (41) to convert tissue data to surgeon usable information. For example, the variable reflectance based on wavelengths with respect to obscuring material may be utilized to identify a critical structure in the anatomy. Moreover, this control system (40) combines the identified spectral signature and the structured light data in an image. For example, this control system (40) may be employed to create a three-dimensional data set for surgical use in a system with augmentation image overlays. Techniques may be employed both intraoperatively and preoperatively using additional visual information. In various instances, this control system (40) is configured to provide warnings to a clinician when in the proximity of one or more critical structures. Various algorithms may be employed to guide robotic automation and semi-automated approaches based on the surgical procedure and proximity to the critical structure(s).

The control system (40) depicted in FIG. 3 is configured to detect the critical structure(s) and provide an image overlay of the critical structure and measure the distance to the surface of the visible tissue and the distance to the embedded/buried critical structure(s). In other instances, this control system (40) may measure the distance to the surface of the visible tissue or detect the critical structure(s) and provide an image overlay of the critical structure.

The control system (40) depicted in FIG. 3 includes a spectral control circuit (42). The spectral control circuit (42) includes a processor (43) to receive video input signals from a video input processor (44). The processor (43) is configured to process the video input signal from the video input processor (44) and provide a video output signal to a video output processor (45), which includes a hyperspectral video-out of interface control (metadata) data, for example. The video output processor (45) provides the video output signal to an image overlay controller (46).

The video input processor (44) is coupled to a camera (47) at the patient side via a patient isolation circuit (48). As previously discussed, the camera (47) includes a solid state image sensor (50). The camera (47) receives intraoperative images through optics (63) and the image sensor (50). An isolated camera output signal (51) is provided to a color RGB fusion circuit (52), which employs a hardware register (53) and a Nios2 co-processor (54) to process the camera output signal (51). A color RGB fusion output signal is provided to the video input processor (44) and a laser pulsing control circuit (55).

The laser pulsing control circuit (55) controls a light engine (56). In some versions, light engine (56) includes any one or more of lasers, LEDs, incandescent sources, and/or interface electronics configured to illuminate the patient's body habitus with a chosen light source for imaging by a camera and/or analysis by a processor. The light engine (56) outputs light in a plurality of wavelengths (λ1, λ2, λ3 . . . λn) including near infrared (NIR) and broadband white light. The light output (58) from the light engine (56) illuminates targeted anatomy in an intraoperative surgical site (59). The laser pulsing control circuit (55) also controls a laser pulse controller (60) for a laser pattern projector (61) that projects a laser light pattern (62), such as a grid or pattern of lines and/or dots, at a predetermined wavelength (λ2) on the operative tissue or organ at the surgical site (59). The camera (47) receives the patterned light as well as the reflected or emitted light output through camera optics (63). The image sensor (50) converts the received light into a digital signal.

The color RGB fusion circuit (52) also outputs signals to the image overlay controller (46) and a video input module (64) for reading the laser light pattern (62) projected onto the targeted anatomy at the surgical site (59) by the laser pattern projector (61). A processing module (65) processes the laser light pattern (62) and outputs a first video output signal (66) representative of the distance to the visible tissue at the surgical site (59). The data is provided to the image overlay controller (46). The processing module (65) also outputs a second video signal (68) representative of a three-dimensional rendered shape of the tissue or organ of the targeted anatomy at the surgical site.

The first and second video output signals (66, 68) include data representative of the position of the critical structure on a three-dimensional surface model, which is provided to an integration module (69). In combination with data from the video output processor (45) of the spectral control circuit (42), the integration module (69) may determine distance (dA) (FIG. 1) to a buried critical structure (e.g., via triangularization algorithms (70)), and that distance (dA) may be provided to the image overlay controller (46) via a video out processor (72). The foregoing conversion logic may encompass a conversion logic circuit (41), intermediate video monitors (74), and a camera (56)/laser pattern projector (61) positioned at surgical site (59).

Preoperative data (75) from a CT or MRI scan may be employed to register or align certain three-dimensional deformable tissue in various instances. Such preoperative data (75) may be provided to an integration module (69) and ultimately to the image overlay controller (46) so that such information may be overlaid with the views from the camera (47) and provided to video monitors (74). Registration of preoperative data is further described herein and in U.S. Pat. Pub. No. 2020/0015907, entitled “Integration of Imaging Data,” published Jan. 16, 2020, for example, which is incorporated by reference herein in its entirety.

Video monitors (74) may output the integrated/augmented views from the image overlay controller (46). On a first monitor (74a), the clinician may toggle between (A) a view in which a three-dimensional rendering of the visible tissue is depicted and (B) an augmented view in which one or more hidden critical structures are depicted over the three-dimensional rendering of the visible tissue. On a second monitor (74b), the clinician may toggle on distance measurements to one or more hidden critical structures and/or the surface of visible tissue, for example.

### D. Exemplary Hyperspectral Identifying Signatures

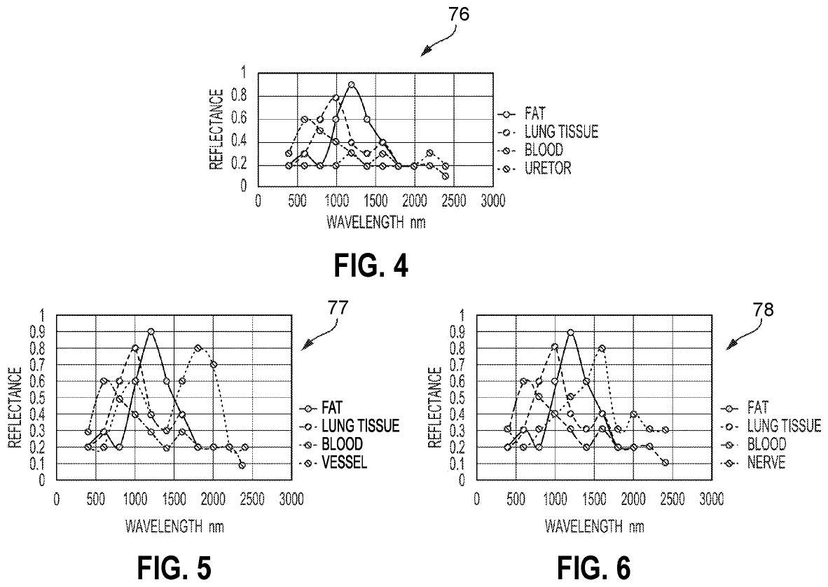

FIG. 4 depicts a graphical representation (76) of an illustrative ureter signature versus obscurants. The plots represent reflectance as a function of wavelength (nm) for wavelengths for fat, lung tissue, blood, and a ureter. FIG. 5 depicts a graphical representation (77) of an illustrative artery signature versus obscurants. The plots represent reflectance as a function of wavelength (nm) for fat, lung tissue, blood, and a vessel. FIG. 6 depicts a graphical representation (78) of an illustrative nerve signature versus obscurants. The plots represent reflectance as a function of wavelength (nm) for fat, lung tissue, blood, and a nerve.

In various instances, select wavelengths for spectral imaging may be identified and utilized based on the anticipated critical structures and/or obscurants at a surgical site (i.e., “selective spectral” imaging). By utilizing selective spectral imaging, the amount of time required to obtain the spectral image may be minimized such that the information may be obtained in real-time, or near real-time, and utilized intraoperatively. In various instances, the wavelengths may be selected by a clinician or by a control circuit based on input by the clinician. In certain instances, the wavelengths may be selected based on machine learning and/or big data accessible to the control circuit via a cloud, for example.

### E. Exemplary Singular EMR Source Emitter Assembly

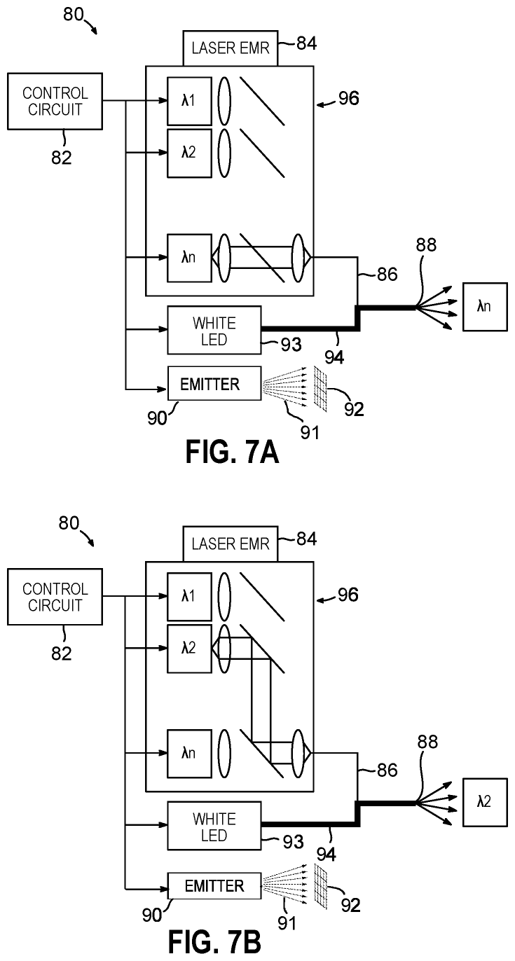

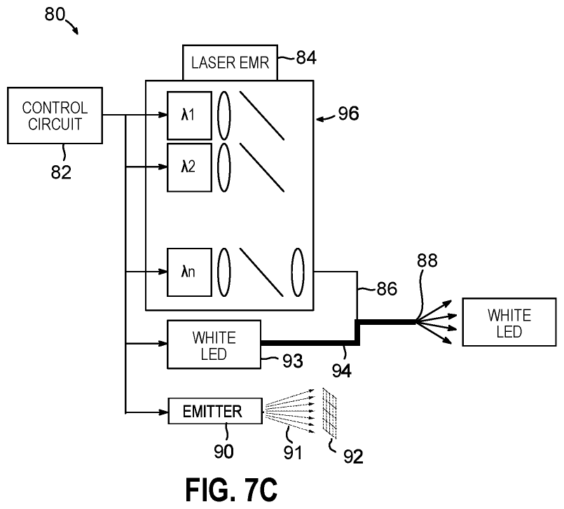

Referring now to FIGS. 7A-7C-, in one aspect, a visualization system (10) includes a receiver assembly (e.g., positioned on a surgical device (16)), which may include a camera (47) including an image sensor (50) (FIG. 3), and an emitter assembly (80) (e.g., positioned on imaging device (17)), which may include an emitter (18) (FIG. 1) and/or a laser light engine (56) (FIG. 3). Further, a visualization system (10) may include a control circuit (82), which may include the control circuit (21) depicted in FIG. 2 and/or the spectral control circuit (42) depicted in FIG. 3, coupled to each of emitter assembly (80) and the receiver assembly. An emitter assembly (80) may be configured to emit EMR at a variety of wavelengths (e.g., in the visible spectrum and/or in the IR spectrum) and/or as structured light (i.e., EMR projected in a particular known pattern) as described below). A control circuit (82) may include, for example, hardwired circuitry, programmable circuitry (e.g., a computer processor coupled to a memory or field programmable gate array), state machine circuitry, firmware storing instructions executed by programmable circuitry, and any combination thereof.

In one aspect, an emitter assembly (80) may be configured to emit visible light, IR, and/or structured light from a single EMR source (84). For example, FIGS. 7A-7C illustrate a diagram of the emitter assembly (80) in alternative states, in accordance with at least one aspect of the present disclosure. In this aspect, the emitter assembly (80) comprises a channel (86) connecting an EMR source (84) to a first emitter (88) configured to emit visible light (e.g., RGB), IR. The channel (86) may include, for example, a fiber optic cable. The EMR source (84) may include, for example, a light engine (56) (FIG. 3) including a plurality of light sources configured to selectively output light at respective wavelengths. In the example shown, the emitter assembly (80) comprises a white LED (93) connected to the first emitter (88) via another channel (94). A second emitter (90) is configured to emit structured light (91) in response to being supplied EMR of particular wavelengths from the EMR source (84). The second emitter (90) may include a filter configured to emit EMR from the EMR source (84) as structured light (91) to cause the emitter assembly (80) to project a predetermined pattern (92) onto the target site.

The depicted emitter assembly (80) further includes a wavelength selector assembly (96) configured to direct EMR emitted from the light sources of the EMR source (84) toward the first emitter (88). In the depicted aspect, the wavelength selector assembly (96) includes a plurality of deflectors and/or reflectors configured to transmit EMR from the light sources of the EMR source (84).

In one aspect, a control circuit (82) may be electrically coupled to each light source of the EMR source (84) such that it may control the light outputted therefrom via applying voltages or control signals thereto. The control circuit (82) may be configured to control the light sources of the EMR source (84) to direct EMR from the EMR source (84) to the first emitter (88) in response to, for example, user input and/or detected parameters (e.g., parameters associated with the surgical instrument or the surgical site). In one aspect, the control circuit (82) is coupled to the EMR source (84) such that it may control the wavelength of the EMR generated by the EMR source (84). In various aspects, the control circuit (82) may control the light sources of the EMR source (84) either independently or in tandem with each other.

In some aspects, the control circuit (82) may adjust the wavelength of the EMR generated by the EMR source (84) according to which light sources of the EMR source (84) are activated. In other words, the control circuit (82) may control the EMR source (84) so that it produces EMR at a particular wavelength or within a particular wavelength range. For example, in FIG. 7A, the control circuit (82) has applied control signals to the nth light source of the EMR source (84) to cause it to emit EMR at an nth wavelength (λn), and has applied control signals to the remaining light sources of the EMR source (84) to prevent them from emitting EMR at their respective wavelengths. Conversely, in FIG. 7B the control circuit (82) has applied control signals to the second light source of the EMR source (84) to cause it to emit EMR at a second wavelength (λ2), and has applied control signals to the remaining light sources of the EMR source (84) to prevent them from emitting EMR at their respective wavelengths. Furthermore, in FIG. 7C the control circuit (82) has applied control signals to the light sources of the EMR source (84) to prevent them from emitting EMR at their respective wavelengths, and has applied control signals to a white LED source to cause it to emit white light.

In addition to the foregoing, at least part of any one or more of the surgical visualization system (10) depicted in FIG. 1, the control system (20) depicted in FIG. 2, the control system (40) depicted in FIG. 3, and/or the emitter assembly (80) depicted in FIGS. 7A and 7B may be configured and operable in accordance with at least some of the teachings of U.S. Pat. Pub. No. 2020/0015925, entitled “Combination Emitter and Camera Assembly,” published Jan. 16, 2020, which is incorporated by reference above. In one aspect, a surgical visualization system (10) may be incorporated into a robotic system in accordance with at least some of such teachings.

### II. Exemplary Endoscope with Source and Pixel Level Image Modulation for Multi Spectral Imaging

In some instances, it may be desirable for a surgical visualization system capture hyperspectral information regarding a surgical field without disturbing illumination that may be used for RGB visualization of the field by a surgeon. In general, accommodating these types of disparate visualization objectives may involve tradeoffs, as the broader the spectrum that is used for illumination, the lower the power of the illumination will be. Aspects of the disclosed technology may address this tradeoff while still allowing for both hyperspectral and RGB visualization of a surgical field.

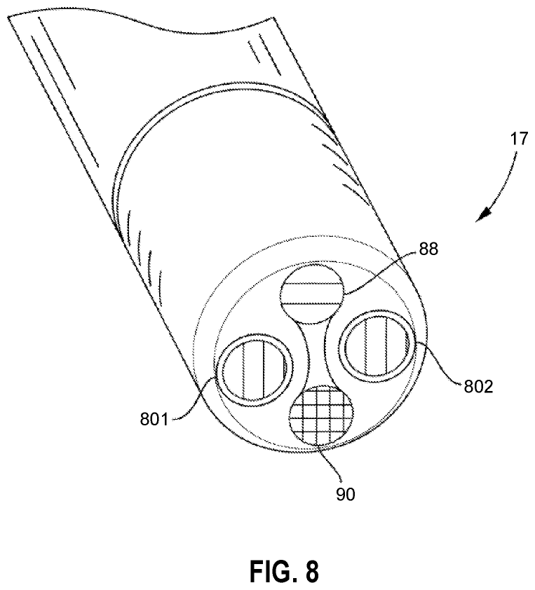

Turning to FIG. 8, that figure illustrates a potential configuration for an imaging device (17) comprising both emitters (88, 90) which may emit structured, hyperspectral and/or visible light, as well as imaging elements (e.g., cameras) (801, 802) which may detect light from the emitters (88, 90) that is reflected off objects in a surgical field. In some implementations, an EMR source (84) may be operable to emit both visible light for RGB imaging, as well as spectral light for identification of structures. For example, in a case where it is desired to distinguish a critical structure (e.g., a nerve) from a background tissue (e.g., collagen), the EMR source (84) may be operable to emit both visible light (e.g., light having wavelengths from about 300 to about 700 nanometers), as well as spectral light at one or more first wavelengths (referred to collectively as λ1) which will be reflected by the critical structure and one or more second wavelengths (referred to collectively as λ2) that will be reflected by the background tissue. In the case where a nerve is to be distinguished from collagen, λ1 may be 860 nm, and λ2 may be 1000 nm. However, other values may also be used.

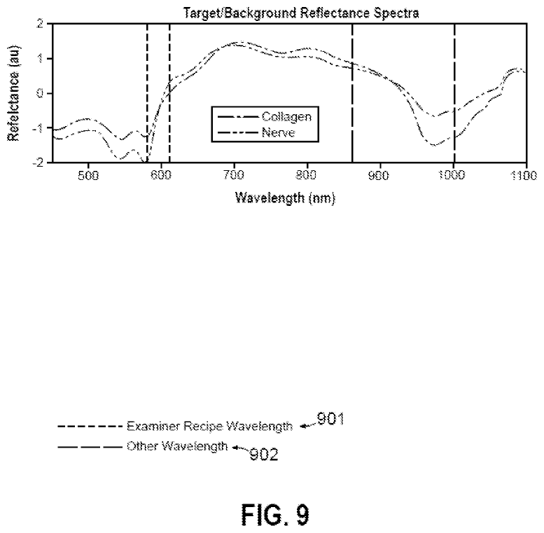

Further examples of how measuring reflectance of various wavelengths of spectral light may enable discrimination between target structures and various types of tissue are provided in FIGS. 4-6, and an additional illustration showing how this same approach can be used to distinguish between nerve and collagen is provided in FIG. 9. With particular focus on FIG. 9, that figure illustrates how there may be multiple wavelengths which may allow for discrimination between a critical structure and background tissue, with the “Examiner Recipe Wavelength” (901) being used to denote wavelengths which may be used in an exemplary implementation, and “Other Wavelength” (902) being used to denote wavelengths which may be used in other implementations. It is also possible that more than more than two wavelengths may be used in some cases. For example, in some cases multiple wavelengths (e.g., four wavelengths) may be combined in a non-linear equation or using a neural network or other machine learning data structure to generate signatures for different types of tissues, and these signatures may be used to identify critical structures or background tissues. Similarly, it is possible that wavelengths other than those specified in the above examples may be used. For instance, in some cases it may be possible that spectral light having other wavelengths between 400 nm and 2000 nm may be used. Accordingly, the above examples should be understood as being illustrative only, and should not be treated as implying limitations on the scope of protection provided by this document or any related document.

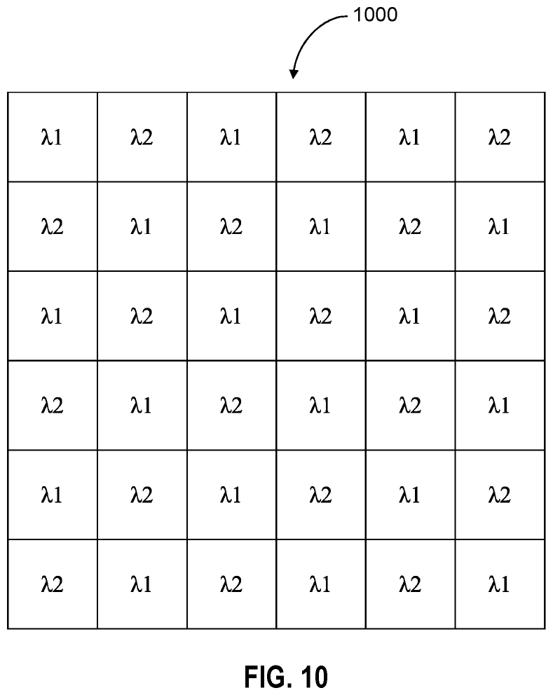

In an implementation such as discussed above in the context of FIG. 8 where there is the potential for emitting both visible light for RGB imaging and spectral light for structure identification, it is possible that hyperspectral and RGB visualization may be harmonized through interleaving the two types of illumination. For example, in a case where there is a target framerate for RGB of 60 frames per second (FPS), the EMR source may be operated in a pulsed manner in which it emits a brief pulse of visible light followed by a brief pulse of light at the spectral wavelengths for identifying the target structure and background tissue (e.g., λ1 and λ2), and cycles through these pulse pairs at the target framerate for the RGB visualization (e.g., in this example, emits a pair of pulses sixty times per second). In this type of scenario, a first camera (801) may be adapted to capture visible light create RGB images, and a second camera (802) may be adapted to capture spectral light for structure identification. In such a case, the second camera (802) may be configured with some pixels adapted to detect light reflected off the background tissue, and some pixels adapted to detect light reflected off of the target structure. An example of this type of pixel configuration is provided in FIG. 10, which depicts an array (1000) of pixels in which alternating pixels would detect spectral light reflected from either the target structure or the background tissue.

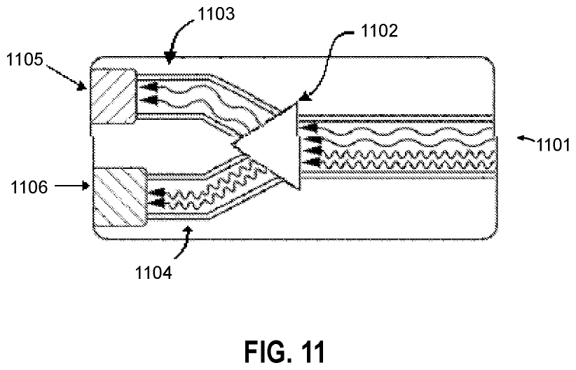

Other approaches to providing single-frame detection of multiple bands of spectral light may also be implemented. For example, as shown in FIG. 11, different wavelengths of spectral light reflected from a surgical site (1101) may be split using a light separator (1102), such as a prism, or, in cases where different wavelengths of light have different polarizations, a polarized beam splitter. This may direct the different wavelengths of spectral light (e.g., e.g., λ1 and λ2) down two optical paths (1103, 1104), leading to separate sets of sensors (1105, 1106), one for identifying a target structure, and one for detecting background tissue. Similarly, in some cases, light used to distinguish between a target structure and background tissue may be detected from reflection of illumination used for capturing visible light images. For example, as shown in FIG. 9, wavelengths of around 580 and 610 nm may be used to identify nerves and collagen, and these wavelengths are within the range of 300 to 700 nm which may be used to provide illumination for visible light images. In this type of case, a camera may be instrumented with tunable filters that would prevent pixels used for detecting spectral light reflected from nerves or collagen from registering illumination at other than their specified wavelength, thereby allowing for spectral light identification of structures without requiring separate spectral illumination of the surgical field.

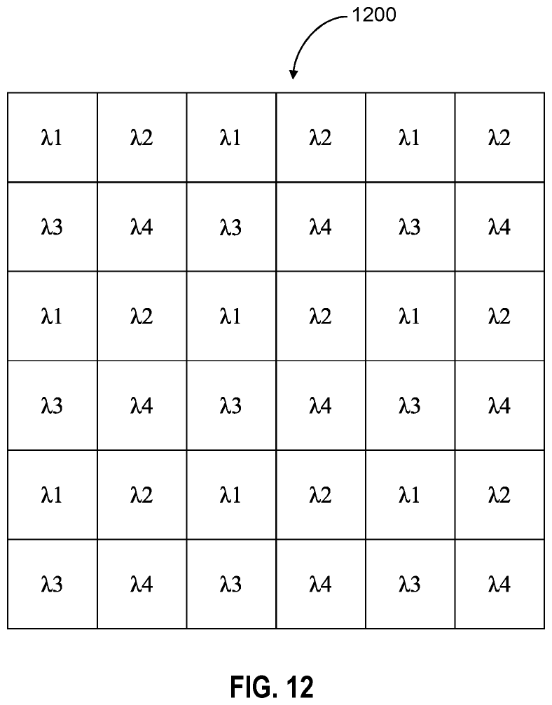

Other types of variation may also be used in different cases. For example, while FIG. 10 illustrated pixels adapted to detect different spectral wavelengths being arranged as an array of alternating pixel types, other arrangements may be possible, such as arrangements where a sensor is split into multiple regions with each region capturing its defined spectral information of a camera's field of view. As another example, while FIG. 10 illustrated an example array having pixels adapted to distinguish one type of structure (e.g., a nerve) from another (e.g., collagen), other arrangements may be used which are adapted to make more find grained distinctions. For example, when distinguishing structures having four characteristics spectral wavelengths, a pixel array (1200) having four classes of pixels such as shown in FIG. 12 may be used. This approach may be expanded to other combinations of wavelengths (e.g., six wavelengths, eight wavelengths) by determining a tiling of the relevant pixel array where each tiling element has at least one pixel for each of the wavelengths to be detected.

Variations in physical configurations of emitters and cameras may also be possible. For example, while FIG. 8 illustrated an example imaging device (17) which included both emitters (88, 90) and cameras (801, 802), in some cases an imaging device (17) may not be configured with both emitters (88, 90) and cameras (801, 802), but instead one or more of those components may be mounted on a different device (e.g., one or more of the emitters (88, 90) may be mounted on a surgical device (16)). Similarly, while the discussion above described an example in which a first camera (801) was used to detect visible light and a second camera (802) was used to detect spectral light, in some cases a single camera may be used to detect both visible and spectral light (e.g., through the user of filters and an array of differentially configured pixels, as described in the context of FIG. 11). Accordingly, the examples described above should be understood as being illustrative only, and should not be treated as implying limitations on the protection provided by this document or any related document.

### III. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A system comprising: (a) an illumination source adapted to simultaneously illuminate a surgical site with spectral light comprising a first wavelength of light and a second wavelength of light; (b) a first set of sensors, the first set of sensors comprising sensors adapted to detect visible light; (c) a second set of sensors, the second set of sensors comprising: (i) sensors adapted to detect the first wavelength of light; and (ii) sensors adapted to detect the second wavelength of light; and (d) a display coupled to a processor configured to display an enhanced image of the surgical site comprising: (i) a visible light image from data detected by the first set of sensors; and (ii) an overlay identifying a target structure based on light detected by the second set of sensors while the surgical site is illuminated by spectral light comprising the first wavelength of light and the second wavelength of light.

### Example 2

The system of Example 1, wherein the system comprises a control circuit adapted to illuminate the surgical site using sets of light pulses, wherein: (a) each set of light pulses comprises a first pulse illuminating the surgical site with visible light, and a second pulse simultaneously illuminating the surgical site with spectral light comprising the first wavelength of light and the second wavelength of light; and (b) the system is adapted to repeatedly illuminate the surgical site with the set of light pulses.

### Example 3

The system of Example 2, wherein the system is adapted to repeatedly illuminate the surgical site with the set of light pulses at a rate of at least thirty times per second.

### Example 4

The system of Example 3, wherein the system is adapted to repeatedly illuminate the surgical site with the set of light pulses at a rate of at least sixty times per second.

### Example 5

The system of any of the preceding Examples, wherein the second set of sensors consists of a plurality of subsets, wherein: (a) each subset is identical and comprises at least one sensor adapted to detect the first wavelength of light, and at least one sensor adapted to detect the second wavelength of light; and (b) the plurality of subsets is disposed as a tiling of a two dimensional imaging area.

### Example 6

The system of any of the preceding Examples, wherein (a) the sensors in the second set of sensors which are adapted to detect the first wavelength of light are configured with filters blocking light not having the first wavelength; and (b) the sensors in the second set of sensors which are adapted to detect the second wavelength of light are configured with filters blocking light not having the second wavelength.

### Example 7

The system of any of the preceding Examples, (a) the sensors adapted to detect the first wavelength of light are disposed on a first optical path; and (b) the sensors adapted to detect the second wavelength of light are disposed on a second optical path.

### Example 8

The system of Example 7, wherein the system comprises a polarized beam splitter disposed at an intersection of the first optical path and the second optical path, and configured to transmit light having a first polarization on the first optical path and light having a second polarization on the second optical path.

### Example 9

The system of any of the preceding Examples, wherein the system comprises an imaging device which comprises both the first set of sensors and the second set of sensors.

### Example 10

The system of any of Examples 1-7, wherein the system comprises an imaging device which comprises the first set of sensors, and a separate surgical device which comprises the second set of sensors.

### Example 11

The system of any of Examples 1 or 5-10, wherein (a) the illumination source is adapted to illuminate the surgical site with broad spectrum light that comprises visible light, light having the first wavelength, and light having the second wavelength; and (b) each sensor from the first set of sensors and the second set of sensors is adapted to detect its respective wavelength of light by a filter which prevents that sensor from detecting light not having its respective wavelength.

### Example 12

The system of any of the preceding Examples, wherein the target structure is from a group consisting of: (a) a nerve; (b) a ureter; (c) an artery; (d) a vein; (e) a common bile duct; (f) a perfusion; (g) a cancerous tumor; and (h) a non-cancerous tumor.

### Example 13

The system of any of the preceding Examples, wherein at least one of: (a) the first wavelength of light; and (b) the second wavelength of light; is between 400 and 2000 nanometers.

### Example 14

The system of any of the preceding Examples, wherein at least one of: (a) the first wavelength of light; and (b) the second wavelength of light; is between 550 and 625 nanometers (nm).

### Example 15

The system of any of Examples 1 through 12, wherein the first wavelength of light and the second wavelength of light are both greater than 800 nm.

### Example 16

The system of any of the preceding Examples, wherein the first set of sensors comprises: (a) sensors adapted to detect green light; (b) sensors adapted to detect blue light; and (c) sensors adapted to detect red light.

### Example 17

The system of any of Examples 1-9 or 11-16, wherein the illumination source, the first set of light sensors and the second set of light sensors are disposed on a distal tip of an endoscope.

### Example 18

The system of any of the preceding Examples, wherein the first wavelength of light corresponds to a spectral signature of the target structure, and the second wavelength of light corresponds to a spectral signature of a background.

### Example 19

A method comprising: (a) simultaneously illuminating a surgical site with spectral light comprising a first wavelength of light and a second wavelength of light; (b) detecting visible light reflected from the interior of the body of the patient with a first set of sensors; (c) simultaneously: (i) detecting, with a second set of sensors, the first wavelength of light from light reflected by the surgical site; and (ii) detecting, with a third set of sensors, the second wavelength of light from light reflected by the surgical site; (d) identifying a location for a target structure based on the light detected by the second set of sensors and the third set of sensors; and (e) displaying an enhanced image of the surgical site comprising a visible light image from data detected by the first set of sensors and an overlay identifying the target structure at the identified location.

### Example 20

The method of Example 19, wherein the method comprises: (a) illuminating the surgical site by emitting a set of light pulses comprising: (i) a first pulse illuminating the surgical site with visible light; and (ii) a second pulse illuminating the surgical site with spectral light comprising the first wavelength of light and the second wavelength of light; and (b) illuminating the surgical site is performed on each repetition of detecting visible light with the first set of sensors, detecting the second wavelength of light with the second set of sensors, and detecting the third wavelength of light with the third set of sensors.

### Example 21

The method of any of Examples 19 through 20, wherein the method comprises: (a) directing the first wavelength of light down a first optical path; and (b) directing the second wavelength of light down a second optical path.

### Example 22

The method of any of Examples 19-21, wherein the method comprises for each sensor from the first set of sensors, the second set of sensors and the third set of sensors, preventing that sensors from detecting light not having a wavelength it is not adapted to detect using a filter.

### Example 23

The method of any of Examples 19-22, wherein the surgical site is inside of a body of a patient.

### Example 24

A non-transitory computer readable medium, having stored thereon instructions operable to cause a surgical visualization system to perform acts comprising: (a) simultaneously illuminating a surgical site with spectral light comprising a first wavelength of light and a second wavelength of light; (b) detecting visible light reflected from the surgical site with a first set of sensors; (c) simultaneously: (i) detecting, with a second set of sensors, the first wavelength of light from light reflected by the surgical site; and (ii) detecting, with a third set of sensors, the second wavelength of light from light reflected by the surgical site; (d) identifying a location for a target structure based on the light detected by the second set of sensors and the third set of sensors; and (e) displaying an enhanced image of the surgical site comprising a visible light image from data detected by the first set of sensors and an overlay identifying the target structure at the identified location.

### IV. Miscellaneous

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. For instance, while many of the illustrative examples focused in cases in which the disclosed technology was utilized laparoscopically, it is contemplated that the disclosed technology may also be used for open procedures, rather than only being applicable in the laparoscopic context. Various suitable ways in which the teachings herein may be combined will be readily apparent to those of ordinary skill in the art in view of the teachings herein. Such modifications and variations are intended to be included within the scope of the claims.

Furthermore, any one or more of the teachings herein may be combined with any one or more of the teachings disclosed in U.S. Pat. App. No. [Atty. Ref. END9342USNP1], entitled “Endoscope with Synthetic Aperture Multispectral Camera Array,” filed on even date herewith; U.S. Pat. App. No. [Atty. Ref. END9345USNP1], entitled “Scene Adaptive Endoscopic Hyperspectral Imaging System,” filed on even date herewith; and/or U.S. Pat. App. No. [Atty. Ref. END9346USNP1], entitled “Stereoscopic Endoscope with Critical Structure Depth Estimation,” filed on even date herewith. The disclosure of each of these U.S. patent applications is incorporated by reference herein.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices described above may be designed to be disposed of after a single use, or they may be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that may penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A system comprising:
(a) an illumination source adapted to simultaneously illuminate a surgical site with spectral light comprising a first wavelength of light and a second wavelength of light;
(b) a first set of sensors, the first set of sensors comprising sensors adapted to detect visible light;
(c) a second set of sensors, the second set of sensors comprising:
(i) sensors adapted to detect the first wavelength of light; and
(ii) sensors adapted to detect the second wavelength of light; and
(d) a display coupled to a processor configured to display an enhanced image of the surgical site comprising:
(i) a visible light image from data detected by the first set of sensors; and
(ii) an overlay identifying a target structure based on light detected by the second set of sensors while the surgical site is illuminated by spectral light comprising the first wavelength of light and the second wavelength of light.

2. The system of claim 1, wherein the system comprises a control circuit adapted to illuminate the surgical site using sets of light pulses, wherein:
(a) each set of light pulses comprises a first pulse illuminating the surgical site with visible light, and a second pulse simultaneously illuminating the surgical site with spectral light comprising the first wavelength of light and the second wavelength of light; and
(b) the system is adapted to repeatedly illuminate the surgical site with the set of light pulses.

3. The system of claim 2, wherein the system is adapted to repeatedly illuminate the surgical site with the set of light pulses at a rate of at least thirty times per second.

4. The system of claim 1, wherein the second set of sensors consists of a plurality of subsets, wherein:
(a) each subset is identical and comprises at least one sensor adapted to detect the first wavelength of light, and at least one sensor adapted to detect the second wavelength of light; and
(b) the plurality of subsets is disposed as a tiling of a two dimensional imaging area.

5. The system of claim 1, wherein:
(a) the sensors in the second set of sensors which are adapted to detect the first wavelength of light are configured with filters blocking light not having the first wavelength; and
(b) the sensors in the second set of sensors which are adapted to detect the second wavelength of light are configured with filters blocking light not having the second wavelength.

6. The system of claim 1, wherein:
(a) the sensors adapted to detect the first wavelength of light are disposed on a first optical path; and
(b) the sensors adapted to detect the second wavelength of light are disposed on a second optical path.

7. The system of claim 6, wherein the system comprises a polarized beam splitter disposed at an intersection of the first optical path and the second optical path, and configured to transmit light having a first polarization on the first optical path and light having a second polarization on the second optical path.

8. The system of claim 1, wherein the system comprises an imaging device which comprises both the first set of sensors and the second set of sensors.

9. The system of claim 1, wherein the system comprises an imaging device which comprises the first set of sensors, and a separate surgical device which comprises the second set of sensors.

10. The system of claim 1, wherein:
(a) the illumination source is adapted to illuminate the surgical site with broad spectrum light that comprises visible light, light having the first wavelength, and light having the second wavelength; and
(b) each sensor from the first set of sensors and the second set of sensors is adapted to detect its respective wavelength of light by a filter which prevents that sensor from detecting light not having its respective wavelength.

11. The system of claim 1, wherein the target structure is from a group consisting of:
(a) a nerve;
(b) a ureter;
(c) an artery;
(d) a vein;
(e) a common bile duct;
(f) a perfusion;
(g) a cancerous tumor; and
(h) a non-cancerous tumor.

12. The system of claim 1, wherein at least one of:
(a) the first wavelength of light; and
(b) the second wavelength of light is between 400 and 2000 nanometers (nm).

13. The system of claim 12, wherein the first wavelength of light and the second wavelength of light are both greater than 800 nm.

14. The system of claim 1, wherein the first set of sensors comprises:
(a) sensors adapted to detect green light;
(b) sensors adapted to detect blue light; and
(c) sensors adapted to detect red light.

15. The system of claim 1, wherein the illumination source, the first set of light sensors and the second set of light sensors are disposed on a distal tip of an endoscope.

16. A method comprising:
(a) simultaneously illuminating a surgical site with spectral light comprising a first wavelength of light and a second wavelength of light;
(b) detecting visible light reflected from the interior of the body of the patient with a first set of sensors;
(c) simultaneously:
(i) detecting, with a second set of sensors, the first wavelength of light from light reflected by the surgical site; and
(ii) detecting, with a third set of sensors, the second wavelength of light from light reflected by the surgical site;
(d) identifying a location for a target structure based on the light detected by the second set of sensors and the third set of sensors; and
(e) displaying an enhanced image of the surgical site comprising a visible light image from data detected by the first set of sensors and an overlay identifying the target structure at the identified location.

17. The method of claim 16, wherein the method comprises:
(a) illuminating the surgical site by emitting a set of light pulses comprising:
(i) a first pulse illuminating the surgical site with visible light; and
(ii) a second pulse simultaneously illuminating the surgical site with spectral light comprising the first wavelength of light and the second wavelength of light; and
(b) illuminating the surgical site by emitting the set of light pulses is performed on each repetition of detecting visible light with the first set of sensors, detecting the second wavelength of light with the second set of sensors, and detecting the third wavelength of light with the third set of sensors.

18. The method of claim 16, wherein the method comprises:
(a) directing the first wavelength of light down a first optical path; and
(b) directing the second wavelength of light down a second optical path.

19. The method of claim 16, wherein the method comprises for each sensor from the first set of sensors, the second set of sensors and the third set of sensors, preventing that sensors from detecting light not having a wavelength it is not adapted to detect using a filter.

20. A non-transitory computer readable medium, having stored thereon instructions operable to cause a surgical visualization system to perform acts comprising:
(a) simultaneously illuminating a surgical site with spectral light comprising a first wavelength of light and a second wavelength of light;
(b) detecting visible light reflected from the surgical site with a first set of sensors;
(c) simultaneously:
(i) detecting, with a second set of sensors, the first wavelength of light from light reflected by the surgical site;
(ii) detecting, with a third set of sensors, the second wavelength of light from light reflected by the surgical site;
(d) identifying a location for a target structure based on the light detected by the second set of sensors and the third set of sensors; and
(e) displaying an enhanced image of the surgical site comprising a visible light image from data detected by the first set of sensors and an overlay identifying the target structure at the identified location.
